(19) United States
(12) Patent Application Publication
(10) Pub. No.: US 2016/0051153 A1
(43) Pub. Date: Feb. 25, 2016

(54) RADIO FREQUENCY IDENTIFICATION (RFID) ENABLED WIRELESS HEART RATE MONITORING SYSTEM

(71) Applicant: Rayan Nabil M. Mously, Melbourne, FL (US)

(72) Inventor: Rayan Nabil M. Mously, Melbourne, FL (US)

(21) Appl. No.: 14/467,755

(22) Filed: Aug. 25, 2014

(51) Int. Cl. A61B 5/0245; A61B 5/11; A61B 5/00; A61B 5/1455

(52) U.S. Cl. CPC A61B 5/0245; A61B 5/1455; A61B 5/1102; A61B 5/742; A61B 5/747; A61B 5/7282; A61B 5/0006

(57) ABSTRACT

Devices and systems for continuous monitoring of the heart rate of a patient diagnosed with or at risk of heart diseases. Wireless communication technologies such as radio frequency identification (RFID), Bluetooth and WiFi are integrated into a heart rate monitoring device to connect it to a communication device, the patient's database at a healthcare institution and to trigger an automatic call for emergency services through the communication device when emergency medical attention is needed.

### GRANT OF NON-EXCLUSIVE RIGHT

This application was prepared with financial support from the Saudia Arabian Cultural Mission, and in consideration therefore the present inventor(s) has granted The Kingdom of Saudi Arabia a non-exclusive right to practice the present invention.

### BACKGROUND

1. Field of the Disclosure

The present invention relates to devices and systems for continuous monitoring of the heart rate of a patient diagnosed with or at risk of heart diseases as well as devices and systems for triggering personal emergency response, transmitting and receiving the patient's medical information via wireless communication technologies.

2. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Heart rate, or heart pulse, is the speed of the heartbeat measured by the number of heartbeats per unit of time—typically beats per minute (bpm). The normal resting adult human rate ranges from 60-100 bpm. Resting heart rate is a key indicator of real-time and long-term health and fitness conditions, for example, heart attack risk, energy levels, metabolic efficiency and athletic endurance. Some people track their heart rates regularly, for example athletes and performers of various types of physical exercise as well as those diagnosed with heart diseases or are at risk. Recently, it has become necessary for certain groups of people to have their heart rates monitored continuously. Apart from the aforementioned group of people with heart problems and are therefore at a higher risk of experiencing heart attacks, social welfare services, hospitals, families and other caregivers of elderly people are striving to provide better healthcare in response to the increasing medical requirements of the growing aging populations in many countries.

A heart rate monitor is a personal monitoring device that allows one to electronically measure his or her heart rate in real time and/or record the heart rate for later study. Methods of measurement by heart rate monitors include electrocardiography (ECG or EKG), pulse oximetry and seismocardiography. Early models of a heart rate monitor consisted of a monitoring box with a set of electrode leads attached to the chest. Nowadays, heart rate monitors usually comprise two elements: a chest strap transmitter and a wrist receiver or cell phone that usually doubles as a watch or a phone. Strapless heart rate monitors have also been developed and they allow the user to simply touch two sensors on a wristwatch display for a few seconds to view their heart rate. Nearly all modern day heart rate monitors are digital with algorithms to measure and compute heart rates at specified intervals.

Advancements in communications and information technologies have helped modern day heart rate monitors to evolve further into sophisticated, multi-functional devices. These developments are based upon the realization that disease and lifestyle management for those at risk of a cardiac arrest or myocardial infarction (heart attack) needs to extend to beyond continuous heart rate monitoring. Moreover, technology can help reduce the financial burden placed on the national healthcare system. Many of the deaths resulting from cardiac arrests or heart attacks would have been preventable if the call for emergency medical attention was more timely. Therefore, people who have suffered a heart attack, suffer from one or more types of heart disease, or are generally at risk, would benefit greatly by using an emergency contact enabled heart rate monitor. Wireless communication technology incorporated in the heart rate monitor also gives information about the real-time location of the patient. Many of these at-risk people are elderly who do not have a person around 24 hours a day to call for emergency in case they go into a cardiac arrest.

In addition, heart disease patients must monitor many variables including medication, weight and heart rate to accurately manage their condition. Just like the automated emergency contact function, wireless technology can eliminate the patients' burden of having to keep track of the variables and factors pertaining to their medical conditions. With wireless technology, heart rate, medical history records and other useful medical information may be stored in a heart rate monitor and the data is linked to remote care doctors, nurses and managers who use the data to detect early warning signs and then provide the patients with medical attention including medication and lifestyle adjustments. Re-hospitalizations can be avoided. Similarly, during an emergency situation, paramedics and medical officers may access the data in the device upon arrival at the location of the patient, gather information about heart rate before and leading to the cardiac arrest/heart attack (event log) as well as the patient's medical history to enable them to make good medical decisions immediately.

Accordingly, it will be advantageous to develop methods and systems for the continuous monitoring of medical status for elderly people and people who are at risk of cardiac distress, so that they are able to manage their conditions with greater dignity, safety, independence and affordability. These methods and systems should also ideally enable patients to call for medical care, and medical professionals to transmit and retrieve their medical information during an emergency situation.

### SUMMARY

In a first aspect, the present invention relates to a wearable device for continuously monitoring a human subject's heart rate, comprising a heart rate monitor, an active, readable RFID tag with storage memory and software for sampling heart rate data, storing and pushing it, an RFID transceiver, a display screen and at least one other wireless communication transponder. The RFID tag stores heart rate data and medical information of the human subject that can be displayed on the display screen. The heart rate monitor in the device may be an electrocardiogram, pulse oximeter and seismocardiogram. The RFID transceiver transmits data on the active RFID tag to a communication device comprising an active RFID reader. In one embodiment, the frequency band of the active RFID tag and the RFID transceiver has a read range of 1 to 100 m. In one embodiment, the frequency band of the active RFID tag and the RFID transceiver is 433 MHz.

In a second aspect, the present invention relates to a system for continuously monitoring a human subject's heart rate and delivering emergency medical service to the subject when the heart rate is abnormal comprising a wearable monitoring device comprising a heart rate monitor, an active, readable RFID tag with storage memory and software for sampling heart rate data, storing and pushing it to an active RFID reader, an RFID transceiver, a display screen and a short-distance wireless communication transponder, a communication device comprising the active RFID reader, global positioning system, the short-distance wireless communication transponder, software preprogrammed with algorithms, maximum and minimum threshold heart rate values over a prescribed duration and software for initiating a call for emergency medical service, a wireless wide area communications network, a database server of a remote healthcare institution and an emergency medical service provider. In this system, the heart rate data of the human subject stored in the memory of the active RFID tag is continuously transmitted to the active RFID reader for data processing in the communication device. The monitoring device is connected to the communication device via the short-distance wireless communication transponder, the communication device is connected to the database server of the remote healthcare institution via the wireless wide area network. When the communication device detects an abnormal heart rate exceeding a maximum threshold value or dropping below a minimum threshold value over a prescribed duration, an automatic call for emergency medical service is initiated to report the abnormal event and the location of the human subject. The emergency medical service provider reaches the human subject and is able to access information about the abnormal event and the human subject's medical history on the display screen of the monitoring device. The emergency medical service provider can also optionally access the database server of the remote healthcare institution via the wireless wide area network. In one embodiment, the system comprises a personal computer comprising an active RFID reader. In one embodiment, the short-distance wireless communication transponder may be Bluetooth or WiFi. The communication device initiates the call for emergency medical service over a cellular network or WiFi.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates a heart rate monitoring system equipped with RFID and at least one other wireless communication transponder according to one embodiment.

FIG. 2 illustrates the five graphical (P, Q, R, S and T) deflections of an electrocardiogram.

FIG. 3 illustrates an exemplary computer system for transfer and processing of heart data and medical information of a patient.

### DETAILED DESCRIPTION OF THE EMBODIMENTS

Referring now to the drawings, wherein like reference numerals designate identical or corresponding parts throughout the several views.

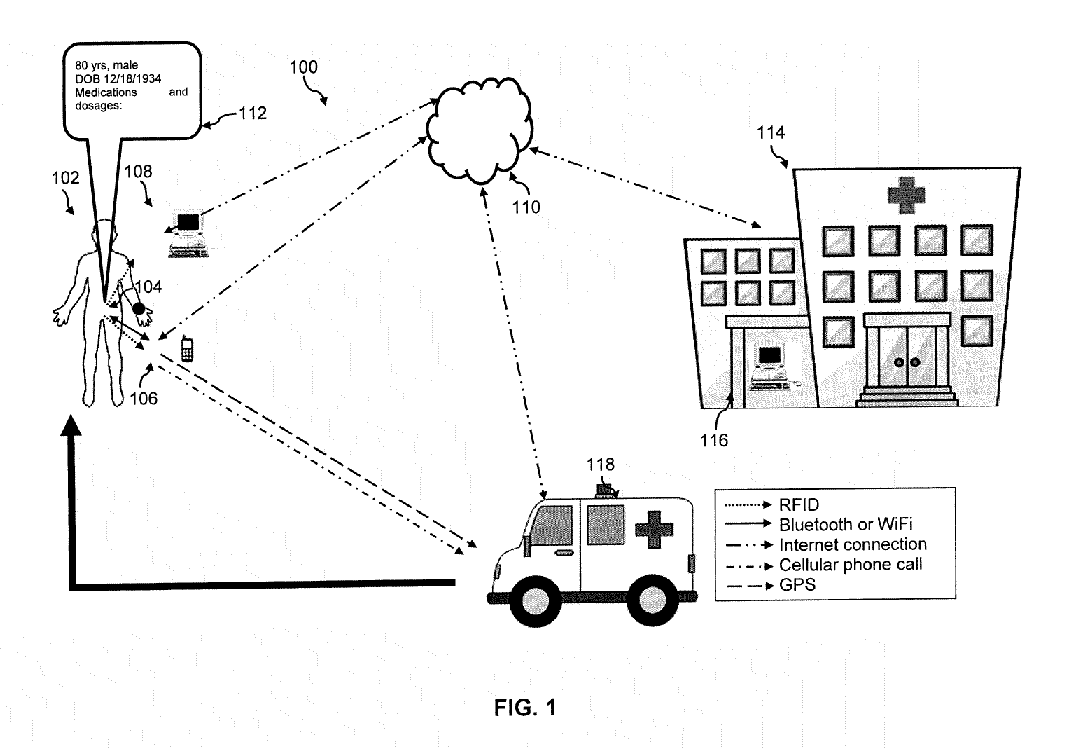

FIG. 1 is a diagram illustrating a heart rate monitoring system 100 in accordance with one embodiment of the present invention. In FIG. 1, patient 102 is a person who is at risk of a cardiac arrest or myocardial infarction due to factors like heart disease diagnosis, diet, age and lifestyle. Due to the aforementioned medical condition, patient 102 wears monitoring device 104 at all times. Therefore, monitoring device is a wearable device, i.e. the device is body-borne under, with or on top of clothing or directly onto the body without requiring clothing. For example, monitoring device 104 may be a wristwatch-like device with straps to attach the device to the wrist or arm, a ring worn around a finger or a biosensor. A biosensor (also called skin sensor) is a small microdermal implant that can be placed anywhere in and/or under the surface of the skin on the body, even discreetly underneath clothing. In one embodiment, the biosensor monitoring device may be in the form of an adhesive patch. To measure heart rate, monitoring device 104 may include an electrocardiogram, a pulse oximeter or a seismocardiogram. For example, in one embodiment, an electrocardiogram within monitoring device 104 measures the heart electrical conduction system of a patient. Electrical impulses generated polarization and depolarization of cardiac tissue are picked up by the electrocardiogram and translated into a waveform. The wave is then used to measure the rate and regularity of heartbeats, and the measured rate in the form of an electrical signal is digitized into heart rate data by a converter included in the design of the monitoring device. A heart rate reading is then displayed on liquid-crystal display (LCD) screen 112. Monitoring device may be configured to measure the heart rate every 5 seconds to every 15 seconds. The electrocardiogram in monitoring device 104 may be wireless wherein electrode leads need not be attached to the chest.

In another embodiment, when a pulse oximetry sensor is used in monitoring device 104, an LED light source reflected from the bloodstream is sensed and heart rate is determined from the pattern and amount of light reflected.

Additionally, monitoring device 104 may include an active radio frequency identification (RFID) chip or tag and antenna or transceiver. Preferably, the RFID tag is ISO 18000-7 active and includes a 433 MHz module. Alternatively, a RFID tag with a 13.56 MHz or a 2.45 GHz module may be included. The RFID tag may have a bandwidth of 10 Mbps and an electronic memory storage capacity of, for example, 1 MB. Heart rate data stored in the RFID tag may be read and transferred to a compatible, active RFID reader or receiver in the patient's mobile communication device 106 and also, optionally a personal computer 108 for data backup storage. The mobile communication device 106 may be a smart phone, cellular phone or personal digital assistant (PDA). Examples of the personal computer include a tablet, laptop and desktop. Heart rate data in monitoring device 104 may be transferred via the RFID to mobile communication device 106 or downloaded onto personal computer 108 and logged in the memory of these devices. The RFID reader in mobile communication device is capable of reading data from the RFID tag in monitoring device 104 1-100 m away therefore it is not necessary that patient 102 carries the mobile communication device at all times but is nevertheless within its vicinity.

Apart from heart rate data, the RFID tag in monitoring device 104 may also store encrypted information about medical prescriptions and medical records of patient 102.

Monitoring device 104 may also be WiFi enabled. Preferably, monitoring device 104 conforms to the Institute of Electrical and Electronics Engineers (IEEE) 802.11a/b/g/n standards. The WiFi technology connects monitoring device 104 to communication device 106. Communication device 106 uses cellular technology such GSM, CDMA and AMPS to connect to network 110. Network 110 may be a wide area network (WAN) and/or the Internet. Network 110 connects monitoring device 104 to patient database server 116 at healthcare institution 114 at a remote location wherein the medical records of patient 102 are stored. Healthcare institution 114 may be a hospital or clinic where patient 102 regularly receives treatment to manage his or her health conditions. Physicians, nurses and medical professionals at healthcare institution through network 110 may monitor and analyze the heart rate data of patient 102 remotely on a regular basis (e.g. weekly), update the patient's records in patient database server 116 and adjust prescribed medications accordingly.

In an alternative embodiment, monitoring device 104 may be Bluetooth enabled and connected to mobile communication device 106 via a Bluetooth transponder. Mobile communication device 106 then connects to patient database server 116 via network 110.

During an emergency, life-threatening situation, for example, cardiac arrhythmia wherein the heart rate of patient 102 is too fast, too slow or irregular, the abnormal heart rate data on the RFID tag on monitoring device 104 may be read by the active RFID reader on mobile communication device 106. When the heart rate exceeds or drops below preprogrammed thresholds (maximum and minimum, respectively), mobile communication device 106 will automatically contact emergency services. Mobile communication device 106 that is also integrated with the global positioning system (GPS) technology will also be able to communicate the real-time location of patient 102 to emergency services.

Once emergency services have been alerted, an emergency medical service provider 118 (e.g. an ambulance) may arrive at the location of patient 102. A paramedic from emergency medical service provider 118 may read the information on the RFID tag displayed on LCD screen 112 of monitoring device 104. The information will help the paramedic to identify patient 102, his or her medical history and medicines currently prescribed taken, as well as the event log (e.g. how long the heart of patient 102 has stopped beating) in order to decide promptly and strategically, what treatment to administer.

It will be readily understood by one skilled in the art that monitoring device 104, mobile communication device 106 and personal computer 108 will each require software programs containing biometric algorithms for the operation of heart rate monitoring system 100. For example, mobile communication device 106 will need software to log the incoming heart rate data from the RFID tag in monitoring device 104 and to automatically contact emergency services if the heart rate of patient 102 drops below or exceeds preprogrammed thresholds for a programmed duration. Furthermore, mobile communication device 106 will also require an interface so that patient 102 or his or her responsible medical professional can set the heart rate thresholds for each biometric measurement to detect changes, whether they are sudden or subtle, and for automatic emergency contact. A software for mobile communication device 106 that handles contacting emergency services over a cellular network or WiFi, will also be desirable. The RFID tag in monitoring device 104, on the other hand, will require embedded software for sampling heart rate data, storing it and pushing it to the active RFID reader in mobile communication device 106 and personal computer 108.

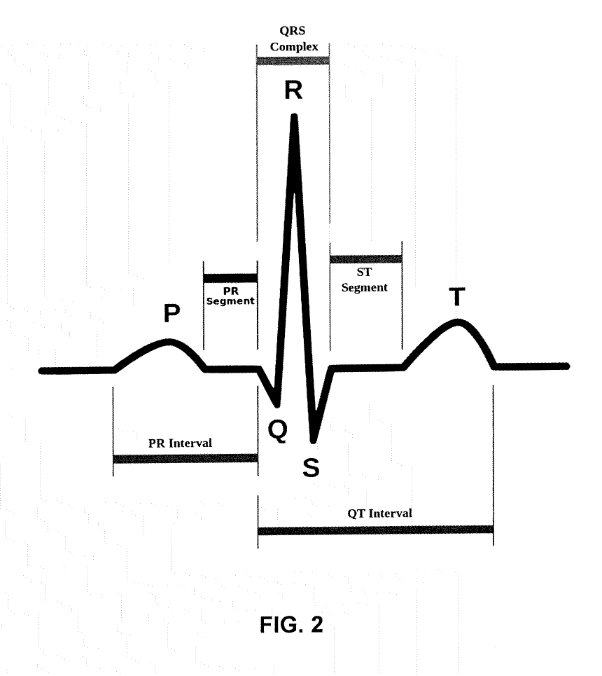

Furthermore, monitoring device 104 includes a software program with one or more algorithms for real-time, automated detection of the QRS complex, for example, the Pan-Tompkins and Hilbert-transform algorithms. Referring to FIG. 2, the QRS complex is the combination of three of the graphical deflections seen on a typical electrocardiogram (ECG). The QRS complex corresponds to the depolarization of the right and left ventricles of the human heart and typically lasts 0.06-0.10 s in a resting adult. In children and during physical activity, the QRS duration may be shorter. Apart from the QRS duration, other important parameters such as the amplitude and the morphology of the QRS complex are also measured and processed by the algorithms included in the monitoring device 104. Such parameters are useful in diagnosing cardiac arrhythmias, conduction abnormalities, ventricular hypertrophy, myocardial infarction, electrolyte derangements and the state of these disorders.

In one embodiment the monitoring device regularly checks the QRS of the wearer for qualitative or quantitative indications of distress or danger. The monitoring device includes a processor or circuitry programmed with operable instructions to regularly obtain and analyze the wearer's heart condition by analysis or observation of the QRS complex. For example, the device may be programmed with a regular QRS complex of the wearer to use as a basis or standard. Over time the device collects and stores or analyzes new QRS information and compares it to the stored values for the standard QRS complex for the wearer. Differences in height, period or shape of the QRS complex can be used as a trigger for sending an alarm or triggering a warning to the wearer. Alternately the QRS can include instructions to store a running average QRS complex or a value derived from the QRS complex. Any significant change (e.g., a change of 5%, 10%, 20%, 30% of any value of the QRS complex) in comparison to the running average or stored standard can then be used as a basis for triggering action.

Monitoring device 104 will also require external power supply, for example, batteries. Acceptable batteries may be disposable, recyclable rechargeable and have a life of at least 48-72 hours. In one embodiment, the power supply may be human-powered piezoelectric batteries.

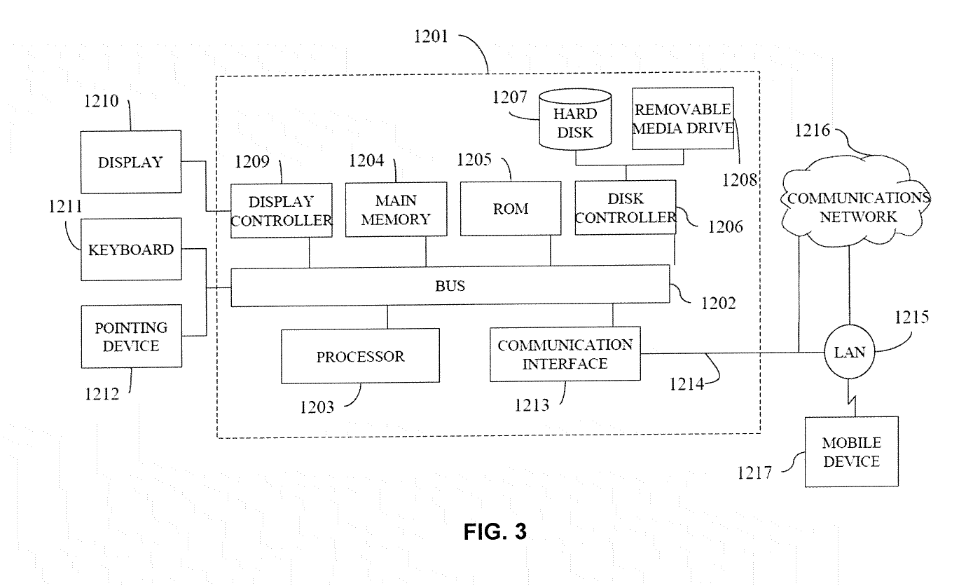

FIG. 3 illustrates an exemplary computer system 1201 for personal computer 108 and patient database server 116 where processing heart rate data from monitoring device 104 may be implemented. The computer system 1201 includes a bus 1202 or other communication mechanism for communicating information, and a processor 1203 coupled with the bus 1202 for processing the information. The computer system 1201 also includes a main memory 1204, such as a random access memory (RAM) or other dynamic storage device (e.g., dynamic RAM (DRAM), static RAM (SRAM), and synchronous DRAM (SDRAM)), coupled to the bus 1202 for storing information and instructions to be executed by processor 1203. In addition, the main memory 1204 may be used for storing temporary variables or other intermediate information during the execution of instructions by the processor 1203. The computer system 1201 further includes a read only memory (ROM) 1205 or other static storage device (e.g., programmable ROM (PROM), erasable PROM (EPROM), and electrically erasable PROM (EEPROM)) coupled to the bus 1202 for storing static information and instructions for the processor 1203.

The computer system 1201 also includes a disk controller 1206 coupled to the bus 1202 to control one or more storage devices for storing information and instructions, such as a magnetic hard disk 1207, and a removable media drive 1208 (e.g., floppy disk drive, read-only compact disc drive, read/write compact disc drive, compact disc jukebox, tape drive, and removable magneto-optical drive). The storage devices may be added to the computer system 1201 using an appropriate device interface (e.g., small computer system interface (SCSI), integrated device electronics (IDE), enhanced-IDE (E-IDE), direct memory access (DMA), or ultra-DMA).

The computer system 1201 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 1201 may also include a display controller 1209 coupled to the bus 1202 to control a display 1210, such as a cathode ray tube (CRT), for displaying information to a computer user. The computer system includes input devices, such as a keyboard 1211 and a pointing device 1212, for interacting with a computer user and providing information to the processor 1203. The pointing device 1212, for example, may be a mouse, a trackball, or a pointing stick for communicating direction information and command selections to the processor 1203 and for controlling cursor movement on the display 1210. In addition, a printer may provide printed listings of data stored and/or generated by the computer system 1201.

The computer system 1201 performs a portion or all of the processing steps of the invention in response to the processor 1203 executing one or more sequences of one or more instructions contained in a memory, such as the main memory 1204. Such instructions may be read into the main memory 1204 from another computer readable medium, such as a hard disk 1207 or a removable media drive 1208. One or more processors in a multi-processing arrangement may also be employed to execute the sequences of instructions contained in main memory 1204. In alternative embodiments, hard-wired circuitry may be used in place of or in combination with software instructions. Thus, embodiments are not limited to any specific combination of hardware circuitry and software.

As stated above, the computer system 1201 includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system 1201, for driving a device or devices for implementing the invention, and for enabling the computer system 1201 to interact with a human user (e.g., print production personnel). Such software may include, but is not limited to, device drivers, operating systems, development tools, and applications software. Such computer readable media further includes the computer program product of the present invention for performing all or a portion (if processing is distributed) of the processing performed in implementing the invention.

The computer code devices of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

The term “computer readable medium” as used herein refers to any medium that participates in providing instructions to the processor 1203 for execution. A computer readable medium may take many forms, including but not limited to, non-volatile media, volatile media, and transmission media. Non-volatile media includes, for example, optical, magnetic disks, and magneto-optical disks, such as the hard disk 1207 or the removable media drive 1208. Volatile media includes dynamic memory, such as the main memory 1204. Transmission media includes coaxial cables, copper wire and fiber optics, including the wires that make up the bus 1202. Transmission media also may also take the form of acoustic or light waves, such as those generated during radio wave and infrared data communications.

Various forms of computer readable media may be involved in carrying out one or more sequences of one or more instructions to processor 1203 for execution. For example, the instructions may initially be carried on a magnetic disk of a remote computer. The remote computer can load the instructions for implementing all or a portion of the present invention remotely into a dynamic memory and send the instructions over a telephone line using a modem. A modem local to the computer system 1201 may receive the data on the telephone line and use an infrared transmitter to convert the data to an infrared signal. An infrared detector coupled to the bus 1202 can receive the data carried in the infrared signal and place the data on the bus 1202. The bus 1202 carries the data to the main memory 1204, from which the processor 1203 retrieves and executes the instructions. The instructions received by the main memory 1204 may optionally be stored on storage device 1207 or 1208 either before or after execution by processor 1203.

The computer system 1201 also includes a communication interface 1213 coupled to the bus 1202. The communication interface 1213 provides a two-way data communication coupling to a network link 1214 that is connected to, for example, a local area network (LAN) 1215, or to another communications network 1216 such as the Internet. For example, the communication interface 1213 may be a network interface card to attach to any packet switched LAN. As another example, the communication interface 1213 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 1213 sends and receives electrical, electromagnetic or optical signals that carry digital data streams representing various types of information.

The network link 1214 typically provides data communication through one or more networks to other data devices. For example, the network link 1214 may provide a connection to another computer through a local network 1215 (e.g., a LAN) or through equipment operated by a service provider, which provides communication services through a communications network 1216. The local network 1214 and the communications network 1216 use, for example, electrical, electromagnetic, or optical signals that carry digital data streams, and the associated physical layer (e.g., CAT 5 cable, coaxial cable, optical fiber, etc). The signals through the various networks and the signals on the network link 1214 and through the communication interface 1213, which carry the digital data to and from the computer system 1201 may be implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 1201 can transmit and receive data, including program code, through the network(s) 1215 and 1216, the network link 1214 and the communication interface 1213. Moreover, the network link 1214 may provide a connection through a LAN 1215 to a mobile device 1217 such as a personal digital assistant (PDA) laptop computer, or cellular telephone.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A wearable device for continuously monitoring a human subject's heart rate, comprising:
a heart rate monitor;
an active, readable RFID tag with storage memory and software for sampling heart rate data, storing and pushing it;
an RFID transceiver;
a display screen; and
at least one other wireless communication transponder;
wherein the RFID tag stores heart rate data and medical information of the human subject that can be displayed on the display screen.

2. The device of claim 1, further comprising one or more QRS detection algorithms.

3. The device of claim 1, wherein the heart rate monitor is selected from the group consisting of electrocardiogram, pulse oximeter and seismocardiogram.

4. The device of claim 1, wherein the RFID transceiver transmits data on the active RFID tag to a communication device comprising an active RFID reader.

5. The device of claim 1, the frequency band of the active RFID tag and the RFID transceiver has a read range of 1 to 100 m.

6. The device of claim 1, wherein the frequency band of the active RFID tag and the RFID transceiver is 433 MHz.

7. The device of claim 1, further comprising an external power supply source.

8. A system for continuously monitoring a human subject's heart rate and delivering emergency medical service to the subject when the heart rate is abnormal, comprising:
a wearable monitoring device comprising a heart rate monitor, an active, readable RFID tag with storage memory and software for sampling heart rate data, storing and pushing it to an active RFID reader, an RFID transceiver, a display screen and a short-distance wireless communication transponder;
a communication device comprising the active RFID reader, global positioning system, the short-distance wireless communication transponder, software preprogrammed with algorithms, maximum and minimum threshold heart rate values over a prescribed duration and software for initiating a call for emergency medical service;
a wireless wide area communications network;
a database server of a remote healthcare institution; and
an emergency medical service provider;
wherein the heart rate data of the human subject stored in the memory of the active RFID tag is continuously transmitted to the active RFID reader for data processing in the communication device;
wherein the monitoring device is connected to the communication device via the short-distance wireless communication transponder, the communication device is connected to the database server of the remote healthcare institution via the wireless wide area network;
wherein the communication device detects an abnormal heart rate exceeding a maximum threshold value or dropping below a minimum threshold value over a prescribed duration and initiates an automatic call for emergency medical service to report the abnormal event and the location of the human subject; and
wherein the emergency medical service provider reaches the human subject, accesses information about the abnormal event and the human subject's medical history on the display screen of the monitoring device and optionally accesses the database server of the remote healthcare institution via the wireless wide area network.

9. The system of claim 8, wherein the algorithms include one or more QRS detection algorithms.

10. The system of claim 7, further comprising a personal computer comprising an active RFID reader.

11. The system of claim 8, wherein the heart rate monitor is selected from the group consisting of electrocardiogram, pulse oximeter and seismocardiogram.

12. The system of claim 8, the frequency band of the active RFID tag, the RFID transceiver and the active RFID reader has a read range of 1 to 100 m.

13. The system of claim 8, wherein the frequency band of the active RFID tag, the RFID transceiver and the active RFID reader is 433 MHz.

14. The system of claim 8, wherein the monitoring device further comprises an external power supply source.

15. The system of claim 8, wherein the short-distance wireless communication transponder is a Bluetooth transponder.

16. The system of claim 8, wherein the short-distance wireless communication transponder is a WiFi transponder.

17. The system of claim 8, wherein the communication device initiates the call for emergency medical service over a cellular network.

18. The system of claim 8, wherein the communication device initiates the call for emergency medical service over WiFi.
